(19) United States
(12) Patent Application Publication
(10) Pub. No.: US 2024/0066068 A1
(43) Pub. Date: Feb. 29, 2024

(54) COMPOSITION FOR SKIN IMPROVEMENT, CONTAINING CULTURE LIQUID OF UMBILICAL-CORD-DERIVED MESENCHYMAL STEM CELLS AS ACTIVE INGREDIENT

(71) Applicant: HANS PHARMA CO., LTD., Seoul (KR)

(72) Inventors: Na Eun LEE, Seongnam-si (KR); Jung Tae LEE, Namyangju-si (KR); Geun Young KIM, Seoul (KR); Jin Young KIM, Seoul (KR); Dong Wook KIM, Seoul (KR); Min Ji LEE, Seoul (KR); Ro Un LEE, Seoul (KR)

(73) Assignee: HANS PHARMA CO., LTD., Seoul (KR)

(21) Appl. No.: 18/268,734

(22) PCT Filed: Nov. 5, 2021
(86) PCT No.: PCT/KR2021/016007

§ 371 Date: Jun. 21, 2023

(30) Foreign Application Priority Data
Dec. 22, 2020 (KR) 10-2020-0180918

(51) Int. Cl. A61K 35/28; A61K 8/98; A61P 17/02; A61P 29/00; A61Q 19/00; A61Q 19/08; C12N 5/0775

(52) U.S. Cl. CPC A61K 35/28; A61K 8/982; A61P 17/02; A61P 29/00; A61Q 19/007; A61Q 19/08; C12N 5/0668

(57) ABSTRACT

The present disclosure relates to a composition for skin improvement and a pharmaceutical composition for preventing or treating an inflammatory skin disease, the compositions including an umbilical cord-derived mesenchymal stem cell culture medium as an active ingredient. The compositions exhibit a wound relief, wrinkle improvement, regeneration, elasticity increase, moisturizing, barrier strengthening, anti-inflammatory or antioxidant effect, and thus can be effectively used as a cosmetic composition for skin improvement or a pharmaceutical composition for preventing or treating an inflammatory skin disease.

### TECHNICAL FIELD

The present disclosure relates to a composition for skin improvement, including an umbilical cord-derived mesenchymal stem cell culture medium as an active ingredient.

### BACKGROUND ART

Stem cells are known to be involved in biological actions by regulating the micro-environment of damaged tissues, such as promoting angiogenesis, suppressing inflammation, and regulating immunity in the human body. These biological actions occur through the secretion of various growth factors, cytokines, the extracellular matrix, and antioxidant proteins that promote the protection and regeneration of damaged tissues, from mesenchymal stem cells. This is called the paracrine effect.

Since a large number of ingredients secreted from mesenchymal stem cells can also be included in a stem cell culture medium, the cosmetics and pharmaceutical industries are making efforts to develop cosmetics and medicines using these stem cell culture medium factors.

Meanwhile, Korean Patent Publication No. 10-2009-0116659 discloses a cosmetic composition for whitening, comprising an umbilical cord-derived mesenchymal stem cell culture medium. However, there is nothing known about the skin improvement effect of an umbilical cord-derived mesenchymal stem cell culture medium through wound relief, wrinkle improvement, regeneration, elasticity increase, moisturizing, barrier strengthening, and anti-inflammation or antioxidation.

### DISCLOSURE

### Technical Problem

An object of the present disclosure is to provide a cosmetic composition for skin improvement, which exhibits a skin improvement effect through wound relief, wrinkle improvement, regeneration, elasticity increase, moisturizing, barrier strengthening, anti-inflammation, or anti-oxidation.

Another object of the present disclosure is to provide a pharmaceutical composition for preventing or treating an inflammatory skin disease.

### Technical Solution

An aspect of the present disclosure provides a cosmetic composition for skin improvement, including an umbilical cord-derived mesenchymal stem cell culture medium as an active ingredient.

The skin improvement may be wound relief, wrinkle improvement, regeneration, elasticity increase, moisturizing, barrier strengthening, anti-inflammation, or antioxidation.

The term “umbilical cord” as used herein may refer to a string connecting the mother body and the belly so that the mammalian fetus can grow in the placenta, and may generally refer to a tissue consisting of three blood vessels surrounded by Wharton's jelly, i.e., two umbilical arteries and one umbilical vein, and is also referred to as the Korean word “Jaedae.”

The term “mesenchymal stem cells” as used herein may refer to stem cells differentiated from the mesoderm resulting from the division of a fertilized egg and present in cartilage, bone tissue, adipose tissue, bone marrow stroma, and the like. Mesenchymal stem cells have the ability to maintain stemness and self-renewal and differentiate into various cells, including chondrocytes, osteoblasts, muscle cells, and adipocytes, and may be extracted from bone marrow, adipose tissue, umbilical cord blood, synovial membrane, trabecular bone, muscle, infrapatellar fat pad, and the like. Mesenchymal stem cells inhibit the activity and proliferation of T lymphocytes and B lymphocytes, inhibit the activity of natural killer cells (NK cells), and have immunomodulatory activity to regulate the functions of dendritic cells and macrophages, and thus are cells capable of allotransplantation and xenotransplantation.

Therefore, the term “umbilical cord-derived mesenchymal stem cells” as used herein may refer to cells derived from umbilical cord or Wharton's jelly tissue of umbilical cord, and having the ability to differentiate into various tissue cells.

The umbilical cord-derived mesenchymal stem cell culture medium may include 6Ckine, adiponectin/Acrp30, angiogenin, angiopoietin-1(ANGPT-1), ANGPT-2, angiopoietin-like 1 (ANGPTL-1), ANGPTL-2, angiostatin, APRIL, artemin, BD-1, BAX, bone morphogenetic protein (BMP)-2, BMP-3, BMP-4, bone morphogenetic protein receptor (BMPR-IA)/anaplastic lymphoma kinase (ALK)-3, C-C chemokine receptor (CCR)1, CCR2, CCR4, CCR6, CCR7, CCR8, CCR9, CD30 ligand/TNFSF8, CD40/TNFRSF5, CD40 ligand/TNFSF6/CD154, Csk, CLC, CRTH-2, CTACK/C-C motif chemokine ligand 27 (CCL27), CXCR1/interleukin 8 receptor alpha (IL-8 RA), CXCR2/interleukin 8 receptor beta (IL-8 RB), CXCR5/BLR-1, EDA-A2, EDG-1, endocrine-gland-derived vascular endothelial growth factor (EG-VEGF)/PK1, endostatin, ErbB4, Fas ligand, FGF Basic (basic fibroblast growth factor), FGF R4, FGF-9, FGF-10/KGF-2, FGF-11, IL-13 1B, growth differentiation factor (GDF)3, GDF5, GDF9, GDF11, GDF-15, GRO-a, heparin-binding EGF (HB-EGF), heme-controlled repressor (HCR)(CRAM-A/B), HRG1-α/NRG1-α, insulin-like growth factor-binding protein (IGFBP)-3, IGFBP-6, IGFBP-related protein (IGFBP-rp)1/IGFBP-7, lymphotoxin-β/TNFSF3, macrophage colony-stimulating factor (M-CSF), MDC, macrophage inflammatory proteins (MIP)-1a, MIP-1b, MIP 2, neutrophil activating protein (NAP)-2, PF4/CXCL4, palate, lung and nasal epithelium clone protein (PLUNC), thrombospondin-1, TIMP-1, TIMP-2, TMEFF1/tomoregulin-1, tumor necrosis factor receptor type 1-associated DEATH domain protein (TRADD), or a combination thereof. For example, the umbilical cord-derived mesenchymal stem cell culture medium may include, among the 71 types of proteins described above, at least two, at least three, at least four, at least five, at least six, at least seven, at least eight, at least nine, at least 10, at least 11, at least 12, at least 13, at least 14, at least 15, at least 16, at least 17, or all of the proteins.

In an embodiment, as a result of measuring ingredients of the umbilical cord-derived mesenchymal stem cell culture medium by using iBright Analysis Software, the ingredients may have signal intensities as shown in Table 1 below.

TABLE 1

| No. | Scretome | Signal intensity |
|---|---|---|
| 1 | 6Ckine | 150,000-200,000 |
| 2 | Adiponectin/Acrp30 | 965,000-1,015,000 |
| 3 | Angiogenin | 265,000-315,000 |
| 4 | Angiopoietin-1 | 745,000-795,000 |
| 5 | Angiopoietin-2 | 1,155,000-1,205,000 |
| 6 | Angiopoietin-like 1 | 145,000-195,000 |
| 7 | Angiopoietin-like 2 | 425,000-475,000 |
| 8 | Angiostatin | 1,085,000-1,235,000 |
| 9 | APRIL | 1,245,000-1,295,000 |
| 10 | Artemin | 95,000-145,000 |
| 11 | BD-1 | 85,000-135,000 |
| 12 | BAX | 1,135,000-1,185,000 |
| 13 | BMP-2 | 295,000-345,000 |
| 14 | BMP-3 | 305,000-355,000 |
| 15 | BMP-4 | 535,000-585,000 |
| 16 | BMPR-IA/ALK-3 | 345,000-395,000 |
| 17 | CCR1 | 335,000-385,000 |
| 18 | CCR2 | 415,000-465,000 |
| 19 | CCR4 | 345,000-395,000 |
| 20 | CCR6 | 485,000-535,000 |
| 21 | CCR7 | 1,245,000-1,295,000 |
| 22 | CCR8 | 1,025,000-1,075,000 |
| 23 | CCR9 | 1,155,000-1,205,000 |
| 24 | CD30 Ligand/TNFSF8 | 555,000-605,000 |
| 25 | CD40/TNFRSF5 | 285,000-335,000 |
| 26 | CD40 Ligand/TNFSF5/CD154 | 655,000-705,000 |
| 27 | Csk | 35,000-85,000 |
| 28 | CLC | 285,000-335,000 |
| 29 | CRTH-2 | 785,000-835,000 |
| 30 | CTACK/CCL27 | 895,000-945,000 |
| 31 | CXCR1/IL-8 RA | 1,035,000-1,085,000 |
| 32 | CXCR2/IL-8 RB | 1,075,000-1,125,000 |
| 33 | CXCR5/BLR-1 | 55,000-105,000 |
| 34 | EDA-A2 | 225,000-265,000 |
| 35 | EDG-1 | 265,000-315,000 |
| 36 | EG-VEGF/PK1 | 235,000-285,000 |
| 37 | Endostatin | 95,000-145,000 |
| 38 | ErbB4 | 195,000-245,000 |
| 39 | Fas Ligand | 85,000-135,000 |
| 40 | FGF Basic | 165,000-215,000 |
| 41 | FGF R4 | 435,000-485,000 |
| 42 | FGF-9 | 1,265,000-1,315,000 |
| 43 | FGF-10/KGF-2 | 115,000-165,000 |
| 44 | FGF-11 | 655,000-705,000 |
| 45 | IL-13 1B | 475,000-525,000 |
| 46 | GDF3 | 145,000-195,000 |
| 47 | GDF5 | 115,000-165,000 |
| 48 | GDF9 | 165,000-215,000 |
| 49 | GDF11 | 95,000-145,000 |
| 50 | GDF-15 | 655,000-705,000 |
| 51 | GRO-a | 215,000-265,000 |
| 52 | HB-EGF | 95,000-1,045,000 |
| 53 | HCR (CRAM-A/B) | 485,000-535,000 |
| 54 | HRG1-alpha/NRG1-alpha | 265,000-315,000 |
| 55 | IGFBP-3 | 145,000-195,000 |
| 56 | IGFBP-6 | 15,000-65,000 |
| 57 | IGFBP-rp1/IGFBP-7 | 1,155,000-1,205,000 |
| 58 | Lymphotoxin beta/TNFSF3 | 175,000-225,000 |
| 59 | M-CSF | 615,000-665,000 |
| 60 | MDC | 415,000-465,000 |
| 61 | MIP-1a | 1,245,000-1,295,000 |
| 62 | MIP-1b | 375,000-425,000 |
| 63 | MIP 2 | 205,000-255,000 |
| 64 | NAP-2 | 165,000-215,000 |
| 65 | PF4/CXCL4 | 65,000-115,000 |
| 66 | PLUNC | 115,000-165,000 |
| 67 | Thrombospondin-1 | 325,000-375,000 |
| 68 | TIMP-1 | 1,075,000-1,125,000 |
| 69 | TIMP-2 | 215,000-265,000 |
| 70 | TMEFF1/Tomoregulin-1 | 1,015,000-1,065,000 |
| 71 | TRADD | 525,000-575,000 |

The umbilical cord-derived mesenchymal stem cell culture medium may include at least one protein selected from the group consisting of adiponectin/Acrp30, ANGPT-1, ANGPT-2, angiostatin, APRIL, CCR7, CCR8, CCR9, CRTH-2, CTACK/CCL27, CXCR1/IL-8 RA, FGF-9, GDF-15, HB-EGF, IGFBP-rp1/IGFBP-7, MIP-1a, and TMEFF1/Tomoregulin-1.

The umbilical cord-derived mesenchymal stem cell culture medium may include at least one protein selected from the group consisting of 6Ckine, ANGPT-2, ANGPTL-1, ANGPTL-2, angiostatin, APRIL, artemin, BD-1, BAX, BMP-3, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF5/CD154, Csk, CLC, CRTH-2, CTACK/CCL27, CXCR1/IL-8 RA, CXCR2/IL-8 RB, CXCR5/BLR-1, EDA-A2, EDG-1, EG-VEGF/PK1, ErbB4, Fas ligand, FGF R4, FGF-9, FGF-10/KGF-2, FGF-11, GDF3, GDF5, GDF9, GRO-a, HCR(CRAM-A/B), HRG1-α/NRG1-α, IGFBP-rp1/IGFBP-7, lymphotoxin-β/TNFSF3, M-CSF, MDC, MIP-1a, MIP-1b, MIP 2, NAP-2, PF4/CXCL4, PLUNC, TMEFF1/Tomoregulin-1, and TRADD, which are not included in the umbilical cord-derived mesenchymal stem cell culture medium of Kangstem Biotech (Seoul, Korea).

The umbilical cord-derived mesenchymal stem cell culture medium may include at least one protein selected from the group consisting of 6Ckine, adiponectin/Acrp30, angiogenin, ANGPT-1, ANGPT-2, ANGPTL-1, ANGPTL-2, angiostatin, APRIL, artemin, BD-1, BAX, BMP-2, BMP-3, BMP-4, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF5/CD154, Csk, CLC, CRTH-2, CTACK/CCL27, CXCR1/IL-8 RA, CXCR2/IL-8 RB, CXCR5/BLR-1, EDG-1, EG-VEGF/PK1, ErbB4, Fas ligand, FGF R4, FGF-9, FGF-10/KGF-2, FGF-11, IL-13 1B, GDF11, HCR(CRAM-A/B), HRG1-α/NRG1-α, IGFBP-rp1/IGFBP-7, lymphotoxin-β/TNFSF3, M-CSF, MDC, MIP-1a, MIP-1b, MIP 2, NAP-2, PF4/CXCL4, PLUNC, TIMP-2, TMEFF1/Tomoregulin-1, and TRADD, which are not included in the neural stem cell culture medium prepared by a method disclosed in Korean Patent Registration No. 10-2172344.

The umbilical cord-derived mesenchymal stem cell culture medium may include: at least one protein selected from the group consisting of 6Ckine, ANGPT-2, ANGPTL-1, ANGPTL-2, angiostatin, APRIL, artemin, BD-1, BAX, BMP-3, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF5/CD154, Csk, CLC, CRTH-2, CTACK/CCL27, CXCR1/IL-8 RA, CXCR2/IL-8 RB, CXCR5/BLR-1, EDA-A2, EDG-1, EG-VEGF/PK1, ErbB4, Fas ligand, FGF R4, FGF-9, FGF-10/KGF-2, FGF-11, GDF3, GDF5, GDF9, GRO-a, HCR(CRAM-A/B), HRG1-α/NRG1-α, IGFBP-rp1/IGFBP-7, lymphotoxin-β/TNFSF3, M-CSF, MDC, MIP-1a, MIP-1b, MIP 2, NAP-2, PF4/CXCL4, PLUNC, TMEFF1/Tomoregulin-1, and TRADD; and at least one protein selected from the group consisting of 6Ckine, adiponectin/Acrp30, angiogenin, ANGPT-1, ANGPT-2, ANGPTL-1, ANGPTL-2, angiostatin, APRIL, artemin, BD-1, BAX, BMP-2, BMP-3, BMP-4, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF5/CD154, Csk, CLC, CRTH-2, CTACK/CCL27, CXCR1/IL-8 RA, CXCR2/IL-8 RB, CXCR5/BLR-1, EDG-1, EG-VEGF/PK1, ErbB4, Fas ligand, FGF R4, FGF-9, FGF-10/KGF-2, FGF-11, IL-13 1B, GDF11, HCR(CRAM-A/B), HRG1-α/NRG1-α, IGFBP-rp1/IGFBP-7, lymphotoxin-β/TNFSF3, M-CSF, MDC, MIP-1a, MIP-1b, MIP 2, NAP-2, PF4/CXCL4, PLUNC, TIMP-2, TMEFF1/Tomoregulin-1, and TRADD.

The cosmetic composition may relieve wounds by restoring the wounds of skin cells.

The cosmetic composition may exhibit a skin wrinkle improvement, regeneration or elasticity increase effect by promoting collagen synthesis in skin cells.

The cosmetic composition may exhibit s skin moisturizing or barrier strengthening effect by promoting aquaporin or hyaluronic acid synthesis.

The term “aquaporin (AQP)” as used herein is an intrinsic membrane protein that induces the passive transport of water molecules by forming a channel in the cell membrane, and refers to a protein that selectively passes only water molecules while restricting the movement of other substances.

The term “hyaluronic acid (HA)” as used herein is a high-molecular compound consisting of N-acetylglucosamine and glucuronic acid, and refers to a factor that helps skin moisturization.

The cosmetic composition may exhibit an antioxidant effect by inhibiting the generation of reactive oxygen species (ROS) in skin cells.

The cosmetic composition may exhibit an anti-inflammatory effect by inhibiting the generation of an inflammatory cytokine in skin cells.

The inflammatory cytokine may be TNF-α, TNF-β, IFN-γ, IL-6, or IL-12, but the present disclosure is not limited thereto. Specifically, the inflammatory cytokine may be TNF-α.

The umbilical cord-derived mesenchymal stem cells may be: i) positive for at least one surface antigen selected from the group consisting of CD44, CD73, CD105, and CD90, and ii) negative for at least one surface antigen selected from the group consisting of CD14, CD19, CD45, and CD34.

The term “positive” as used herein may mean that, in relation to a stem cell surface marker, the surface marker is present in a larger amount or at a higher concentration compared to other non-stem cells as a reference. That is, since any surface marker is present on the surface of a cell, the cell is positive for the marker when the cell can be distinguished from one or more other cell types by using the marker. It may also mean that the cell is expressing the marker in an amount sufficient to generate a signal, e.g., a signal of a cytometer, at a value greater than the background value. For example, a cell may be detectable with an antibody specific for CD44, a stem cell-specific surface antigen, and when the signal from this antibody is detectably greater than that of a control (e.g., a background value), the cell is “CD44⁺.”

The term “negative” as used herein means that, even when an antibody specific for a specific cell surface marker is used, the marker is not detectable compared to the background value. For example, when a cell cannot be detectably labeled with an antibody specific for CD14, the cell is “CD14⁻.”

The immunological properties may be determined by conventional methods known in the art. For example, various methods such as flow cytometry, immunocytochemical staining, or RT-PCR may be used.

The umbilical cord-derived mesenchymal stem cell culture medium may be prepared by a method including the steps of: a) isolating mesenchymal stem cells from umbilical cord from which blood vessels are removed; b) subculturing the isolated mesenchymal stem cells 1 to 10 times in serum-free cell culture medium; and c) filtering after culture medium is obtained in the subculturing step.

As the umbilical cord, a placenta separated after childbirth from a healthy mother (e.g., a mother who is negative for HIV, HCV, or HBV) may be used. That is, the “separated umbilical cord” may mean an umbilical cord that is separated from the mothers body after childbirth. The separated umbilical cord may be rapidly stored in a sterilized container and ice immediately after separation.

A method of separating and obtaining the umbilical cord from the placenta may include, for example, the steps of: separating the umbilical cord from the separated placenta; removing blood outside the separated umbilical cord; removing the arteries and veins of the umbilical cord from which the blood has been removed; and/or finely cut the umbilical cord from which the arteries and veins are removed, to a certain size (e.g., 1 mm to 20 mm). For the removal of the blood, for example, Ca/Mg free DPBS or CaMg free DPBS containing gentamicin may be used.

Next, a step of isolating mesenchymal stem cells from the finely cut umbilical cord (e.g., the separated umbilical cord) by treatment with a separase may be performed. The separase may include collagenase, trypsin, and/or dispase.

Next, the method may include a step of subculturing the isolated umbilical cord-derived mesenchymal stem cells 1 to 10 times as P0. Specifically, the subculturing may be performed three times or four times.

The umbilical cord-derived mesenchymal stem cell culture medium according to the present disclosure may be obtained through the step of filtering after a culture medium is obtained in the subculturing step.

The cosmetic composition may be formulated as a cosmetic formulation commonly prepared in the art, if necessary.

The cosmetic composition may be formulated as, for example, a solution, a suspension, an emulsion, a paste, a gel, a cream, a lotion, powder, a soap, a surfactant-containing cleansing, oil, powder foundation, emulsion foundation, wax foundation, and spray, but the present disclosure is not limited thereto. Specifically, the cosmetic composition may be formulated in the form of skin toner, nutrition lotion, nutrition cream, massage cream, essence, eye cream, cleansing cream, cleansing foam, cleansing water, pack, spray, or powder. In addition, for paste, cream, or gel formulations, the cosmetic composition may include a carrier ingredient selected from the group consisting of animal oil, vegetable oil, wax, paraffin, starch, tragacanth, a cellulose derivative, polyethylene glycol, silicon, bentonite, silica, talc, zinc oxide, and a mixture thereof.

In addition, for solution or emulsion formulations, the cosmetic composition may include a carrier ingredient selected from the group consisting of a solvent, a solubilizing agent, an emulsifying agent, and a mixture thereof. Examples thereof may include water, ethanol, isopropanol, ethyl carbonate, ethyl acetate, benzyl alcohol, benzyl benzoate, propylene glycol, 1,3-butylglycol oil, glycerol aliphatic ester, polyethylene glycol, sorbitan fatty acid ester, and a mixture thereof.

In addition, for suspension formulations, the cosmetic composition may include a carrier ingredient selected from the group consisting of: a liquid diluent such as water, ethanol, or propylene glycol; a suspending agent such as ethoxylated isostearyl alcohol, polyoxyethylene sorbitol ester, and polyoxyethylene sorbitan ester; microcrystalline cellulose; aluminum metahydroxide; bentonite; agar; tragacanth; and a mixture thereof.

The carrier ingredient may be included in an amount of about 1 wt % to about 99.99 wt %, preferably about 80 wt % to about 90 wt %, with respect to the total weight of the cosmetic composition.

Another aspect of the present disclosure provides a pharmaceutical composition for preventing or treating an inflammatory skin disease, including an umbilical cord-derived mesenchymal stem cell culture medium as an active ingredient.

The umbilical cord-derived mesenchymal stem cell culture medium is the same as described above.

The inflammatory skin disease may be at least one disease selected from the group consisting of atopic dermatitis, allergic dermatitis, contact dermatitis, acne, seborrheic dermatitis, sweat band, urticaria, psoriasis, skin sclerosis, eczema, vitiligo, loops, and circular hair loss, but the present disclosure is not limited thereto.

The pharmaceutical composition may include, as an active ingredient, the umbilical cord-derived mesenchymal stem cell culture medium in an amount of about 0.1 wt % to about 90 wt %, particularly about 0.5 wt % to about 75 wt %, and more particularly, about 1 wt % to about 50 wt %, with respect to the total weight of the composition.

The pharmaceutical composition may include conventional and non-toxic pharmaceutically acceptable additives formulated into a preparation according to a general method. For example, the pharmaceutical composition may further include a pharmaceutically acceptable carrier, a diluent, or an excipient.

The pharmaceutical composition may be applied to the skin. The formulation of the pharmaceutical composition may be a formulation for external application to the skin. The formulation for external application to the skin is not particularly limited thereto, but may be prepared in the form of, for example, an ointment, a lotion, a spray, a patch, a cream, an acid, a suspending agent, a poultice, or a gel.

Redundant descriptions are omitted in consideration of the complexity of the present specification, and the terms have the meanings commonly used in the art to which the present disclosure pertains, unless otherwise defined herein.

### Advantageous Effects

A cosmetic composition including an umbilical cord-derived mesenchymal stem cell culture medium, according to an aspect of the present disclosure, exhibits a wound relief, wrinkle improvement, regeneration, elasticity increase, moisturizing, barrier strengthening, anti-inflammatory or antioxidant effect, and thus can be effectively used in a cosmetic composition for skin improvement.

A pharmaceutical composition including an umbilical cord-derived mesenchymal stem cell culture medium, according to another aspect, has the effect of suppressing the production of inflammatory cytokines, and thus can be widely used as a composition for the prevention or treatment of an inflammatory skin disease.

### DESCRIPTION OF DRAWINGS

FIG. 1 is a microscopic image of umbilical cord-derived mesenchymal stem cells at a magnification of ×40 according to an embodiment.

FIG. 2 is a microscopic image of umbilical cord-derived mesenchymal stem cells at a magnification of ×100 according to an embodiment.

FIG. 3 illustrates the result of confirming the osteogenic differentiation potential of umbilical cord-derived mesenchymal stem cells according to an embodiment.

FIG. 4 illustrates the result of confirming the adipogenic differentiation potential of umbilical cord-derived mesenchymal stem cells according to an embodiment.

FIG. 5 illustrates the results of confirming whether stem cell-specific surface markers are expressed in umbilical cord-derived mesenchymal stem cells according to an embodiment through FACS analysis, which is a flow cytometer.

FIG. 6 illustrates the results of confirming whether stem cell-specific surface markers are expressed in umbilical cord-derived mesenchymal stem cells according to an embodiment through immune cell fluorescence staining.

FIG. 7 illustrates the analysis results of protein ingredients included in an umbilical cord-derived mesenchymal stem cell culture medium according to an embodiment.

FIG. 8 is a graph showing the results of measuring the expression intensity of proteins secreted from an umbilical cord-derived mesenchymal stem cell culture medium according to an embodiment.

FIG. 9 illustrates the results of measuring the cell growth rate of human epidermal cells (HaCaT) according to the treatment concentration of an umbilical cord-derived mesenchymal stem cell culture medium according to an embodiment.

FIG. 10 illustrates the results of measuring the cell growth rate of human dermal fibroblasts (HS68) according to the treatment concentration of an umbilical cord-derived mesenchymal stem cell culture medium according to an embodiment.

FIG. 11 illustrates the results of observing the cell morphology of human epidermal cells (HaCaT) according to treatment with an umbilical cord-derived mesenchymal stem cell culture medium according to an embodiment, adipose-derived mesenchymal stem cells, and a bone marrow-derived mesenchymal stem cell culture medium, in which AD denotes adipose, BM denotes bone marrow, and UC denotes umbilical cord.

FIG. 12 illustrates the results of measuring the cell growth rate of human epidermal cells (HaCaT) according to treatment with an umbilical cord-derived mesenchymal stem cell culture medium according to an embodiment, adipose-derived mesenchymal stem cells, and a bone marrow-derived mesenchymal stem cell culture medium, in which AD denotes adipose, BM denotes bone marrow, and UC denotes umbilical cord.

FIG. 13 illustrates microscopic images showing a wound recovery effect and the results of measuring a wound recovery rate, in human epidermal cells (HaCaT) according to the treatment concentration of an umbilical cord-derived mesenchymal stem cell culture medium according to an embodiment.

FIG. 14 illustrates microscopic images showing a wound recovery effect and the results of measuring a wound recovery rate, in human dermal fibroblasts (HS68) according to the treatment concentration of an umbilical cord-derived mesenchymal stem cell culture medium according to an embodiment.

FIG. 15 illustrates microscopic images showing wound recovery and the results of measuring a wound recovery rate, in human epidermal cells (HaCaT) according to treatment with an umbilical cord-derived mesenchymal stem cell culture medium (UC-MSC) according to an embodiment, adipose-derived mesenchymal stem cells (AD-MSC), and a bone marrow-derived mesenchymal stem cell culture medium (BM-MSC).

FIG. 16 illustrates electrophoretic images confirming the expression of COL1A1 and a graph comparing the expression levels of COL1A1, after human dermal fibroblasts (HS68) were treated with an umbilical cord-derived mesenchymal stem cell culture medium according to an embodiment at different concentrations.

FIG. 17 illustrates electrophoretic images confirming the expression of COL3A1 and a graph comparing the expression levels of COL3A1, after human dermal fibroblasts (HS68) were treated with an umbilical cord-derived mesenchymal stem cell culture medium according to an embodiment at different concentrations.

FIG. 18 is a graph comparing the expression levels of PICP in human dermal fibroblasts (HS68) according to the treatment concentration of an umbilical cord-derived mesenchymal stem cell culture medium according to an embodiment.

FIG. 19 illustrates electrophoretic images confirming the expression of AQP3, HAS2, and HAS3 and graphs comparing the expression levels thereof, in human epidermal cells (HaCaT) according to the treatment concentration of an umbilical cord-derived mesenchymal stem cell culture medium according to an embodiment.

FIG. 20 illustrates electrophoretic images confirming the expression of AQP3, HAS2, and HAS3 and a graph comparing the expression levels thereof, in human epidermal cells (HaCaT) according to treatment with an umbilical cord-derived mesenchymal stem cell culture medium according to an embodiment, adipose-derived mesenchymal stem cells (AD), and a bone marrow-derived mesenchymal stem cell culture medium (BM).

FIG. 21 illustrates electrophoretic images showing the expression of TNF-α and a graph comparing the expression levels of TNF-α, after murine macrophages (Raw 264.7) were treated with lipopolysaccharide (LPS) to induce an inflammatory response, and treated with an umbilical cord-derived mesenchymal stem cell culture medium according to an embodiment at different concentrations.

FIG. 22 illustrates: the results (left) of comparing Trolox equivalent antioxidant capabilities in human dermal fibroblasts (HS68) according to the treatment concentration of an umbilical cord-derived mesenchymal stem cell culture medium according to an embodiment; and the results (right) of comparing Trolox equivalent antioxidant capabilities in human dermal fibroblasts (HS68) according to treatment with an umbilical cord-derived mesenchymal stem cell culture medium (UC), an adipose-derived mesenchymal stem cell culture medium (AD), and a bone marrow-derived mesenchymal stem cell culture medium (BM).

FIG. 23 illustrates: the results of measuring changes in concentration of intracellular reactive oxygen species (ROS) in human dermal fibroblasts (HS68) according to the treatment concentration of an umbilical cord-derived mesenchymal stem cell culture medium according to an embodiment; and a graph comparing DCF-DA fluorescence values.

### MODE FOR DISCLOSURE

Hereinafter, the present disclosure will be described in further detail with reference to the following examples. However, these examples are provided for illustrative purposes only and are not intended to limit the scope of the present disclosure.

### Preparation Example 1. Preparation of Umbilical Cord-Derived Mesenchymal Stem Cells and Umbilical Cord-Derived Mesenchymal Stem Cell Culture Medium

An umbilical cord provided during delivery of a healthy mother was washed with phosphate buffered saline (PBS) in a cell culture dish on ice in a clean bench or a biological safety cabinet (BSC). Blood vessels in the umbilical cord were first removed with sterilized scissors, followed by finely cutting to a size of about 3 mm to about 5 mm. The finely cut umbilical cord tissues were transferred to a cell culture flask, followed by treatment with a trypsin enzyme to allow a reaction to occur at 37° C. for 30 minutes, and MEM-alpha (GIBCO) medium containing 5% human platelet lysate (HPL) (Helios U1traGRO), 1% penicillin/streptomycin (P/S) (GIBCO) was added thereto and the tissues were cultured in an incubator at 37° C., to thereby obtain umbilical cord-derived mesenchymal stem cells.

The obtained mesenchymal stem cells were subcultured three times or four times, and then, when the confluency of the cells reached 70% to 80%, the culture medium was replaced with phenol-red free MEM-alpha containing 5% HPL and 1% P/S, followed by culturing for 48 hours, to separate a culture medium. The separated culture medium was filtered with a 0.22 μm filter to obtain an umbilical cord-derived mesenchymal stem cell culture medium.

### Comparative Example 1. Preparation of Adipose-Derived Mesenchymal Stem Cell Culture Medium and Bone Marrow-Derived Mesenchymal Stem Cell Culture Medium

Adipose-derived mesenchymal stem cells (Cat #C-12978) purchased from Promocell and bone marrow-derived mesenchymal stem cells (Cat #PT-2501) purchased from LonZa were subcultured three times or four times, followed by addition of MEM alpha medium containing 5% HPL and 1% P/S, and were further cultured. When the confluency of the cells reached 70% to 80%, the culture medium was replaced with phenol-red-free MEM-alpha, and while being cultured for 48 hours, the adipose-derived mesenchymal stem cells and the bone marrow-derived mesenchymal stem cell culture medium were obtained.

### Experimental Example 1. Characterization of Umbilical Cord-Derived Mesenchymal Stem Cells

### Experimental Example 1.1. Observation of Cell Morphology of Umbilical Cord-Derived Mesenchymal Stem Cells

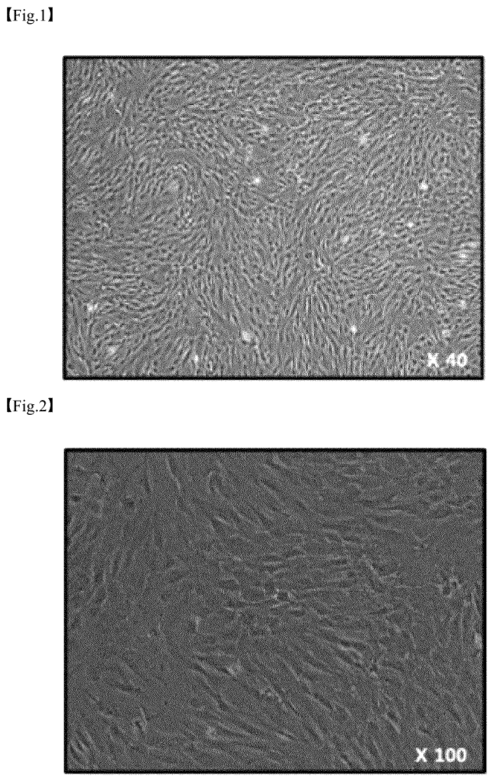

The morphology of the umbilical cord-derived mesenchymal stem cells obtained in Preparation Example 1 was observed under a microscope. FIG. 1 is an image observed under a microscope at a magnification of ×40, and FIG. 2 is an image observed under a microscope at a magnification of ×100.

### Experimental Example 1.2. Analysis of Differentiation Potential of Umbilical Cord-Derived Mesenchymal Stem Cells

To analyze the osteogenic and adipogenic differentiation potentials of the umbilical cord-derived mesenchymal stem cells obtained in Preparation Example 1, the following experiments were carried out.

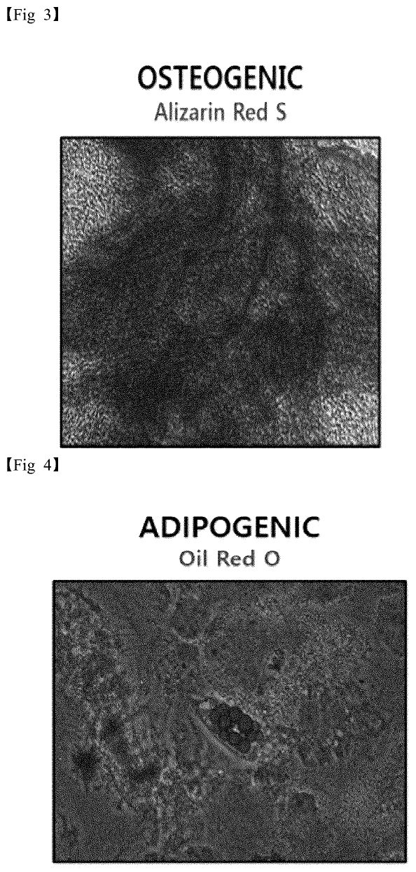

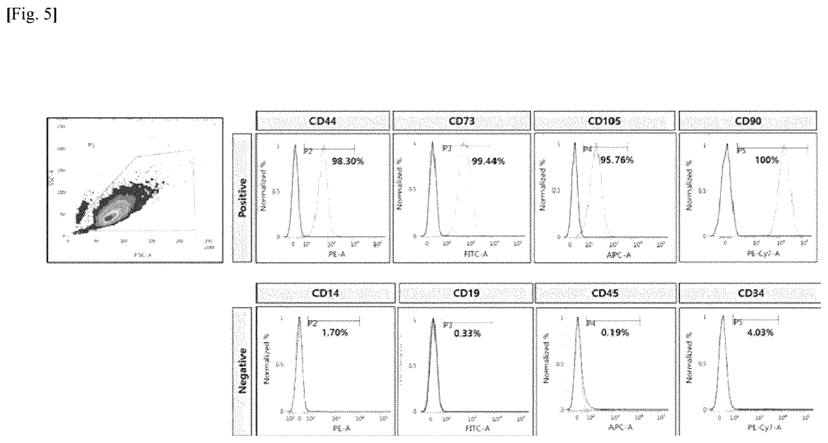

First, to analyze the osteogenic differentiation potential, the umbilical cord-derived mesenchymal stem cells were seeded into a 6-well plate at a density of 2.5×10⁵ cells/well, and then cultured for 24 hours in a low-glucose DMEM medium (containing 10% fetal bovine serum (FBS) and 1% P/S). Thereafter, the DMEM medium was replaced with a complete differentiation medium containing 0.1 μM dexamethasone (Sigma D4902), 10 μM β-glycerol phosphate (Sigma G9891), and 0.25 mM ascorbic acid (AA) (Sigma A4544), followed by culture for 21 days. After the culture was completed, Alizarin Red S staining was performed to confirm whether or not osteocytes are formed. As a result, most of the cells were stained red, through which it was confirmed that the umbilical cord-derived stem cells were differentiated into osteocytes (FIG. 3).

Next, to analyze the adipogenic differentiation potential, the umbilical cord-derived mesenchymal stem cells were seeded into a 6-well plate at a density of 1×10⁵ cells/well, and then cultured for 24 hours in low-glucose DMEM medium (containing 10% FBS and 1% AA). Then, the DMEM medium was replaced with a complete differentiation medium containing 0.5 mM 3-isobutyl-1-methylxanthine (IBMX, Sigma 17018), 1 μM hydrocortisone (Sigma H0888), and 0.1 mM indomethacin (Sigma 17378), followed by culture for 21 hours, and the medium was replaced every 2-3 days. After the culture was completed, Oil Red O (Sigma) staining was performed to confirm the formation of lipid droplets. As a result, large and small substances (fat) that looked like water droplets were stained red, through which it was confirmed that the umbilical cord-derived stem cells were differentiated into adipocytes.

Through these results, it can be confirmed that the umbilical cord-derived mesenchymal stem cells according to an embodiment of the present disclosure have osteogenic and adipogenic differentiation potentials.

### Experimental Example 1.3. Analysis of Surface Marker Expression of Umbilical Cord-Derived Mesenchymal Stem Cells

To analyze whether the umbilical cord-derived mesenchymal stem cells obtained in Preparation Example 1 express stem cell surface markers, the following experiments were carried out.

### Experimental Example 1.3.1. Analysis Through Flow Cytometry

When the confluency of the umbilical cord-derived mesenchymal stem cells obtained in Preparation Example 1 reached 80% to 90%, the culture medium was removed, followed by washing with PBS. Then, trypsin was added to dissociate the cells, and the cells were further washed with PBS. The number of the cells was counted and fluorescence-activated cell sorter (FACS) buffer (PBS+2% FBS) was added to make 1×10⁶ cells/mL, and then the cells were allowed to react with positive markers, i.e., CD44(PE), CD73(FITC), CD105(APC), and CD90(PE-Cy7) and negative markers, i.e., CD14(PE), CD19(FITC), CD45(APC), and CD34(PE-cy7) antibodies. Thereafter, FACS was used to identify specific expression markers of the umbilical cord-derived mesenchymal stem cells. As a result, it was confirmed that the umbilical cord-derived mesenchymal stem cells were selectively positive for CD44, CD73, CD105, and CD90, and selectively negative for CD14, CD19, CD45, and CD34.

### Experimental Example 1.3.2. Analysis Through Immunocytochemical Staining

The umbilical cord-derived mesenchymal stem cells obtained in Preparation Example 1 that were maintained on a 4-well chamber slide were fixed using 4% p-formaldehyde at 37° C. for 20 minutes, and then washed twice with PBS containing calcium ions and magnesium ions. Then, Triton X-100 as a surfactant was diluted to 0.1% in PBS, followed by treatment therewith for 10 minutes, and the cells were washed again with PBS. To prevent non-specific antibodies from being attached and detected, bovine serum albumin (BSA) was diluted to 5% in 0.1% Triton X-100/PBS, and then added and allowed to react with a sample for 1 hour.

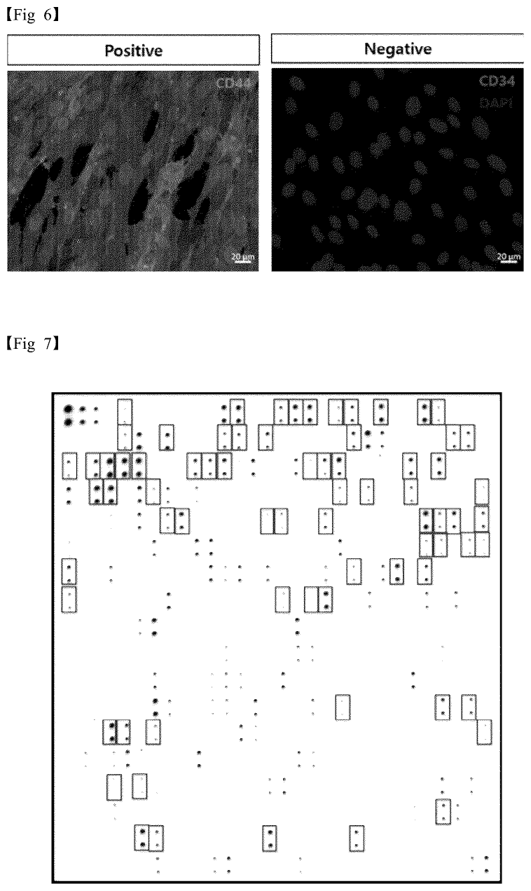

The types of antibodies to be attached vary depending on cells, and the target antibody and dilution ratio for each protein are shown in Table 2 below. A reaction was allowed to occur in a shaker together with the diluted antibody solution at 4° C. for 16 hours. In addition, nuclei were stained using DAPI (abcam, cat.no.ab104139, diluted 1,000 times). The stained sample was imaged using a fluorescence microscope. As a result, it was confirmed that the umbilical cord-derived mesenchymal stem cells expressed CD44 (green), which is a positive stem cell surface marker, and did not express CD34 (red), which is a negative stem cell surface marker (FIG. 6).

TABLE 2

| Antibody | Purchase place and dilution ratio |
|---|---|
| Recombinant Anti-CD44 antibody | Abcam (ab194987), 1/50 |
| Immunocytochemistry/ | Abcam (ab81289), 1/200 |
| Immunofluorescence- | |
| Anti-CD34 antibody | |

Through these results, it can be confirmed that the umbilical cord-derived mesenchymal stem cells according to an embodiment of the present disclosure exhibit stem cell-specific characteristics.

### Experimental Example 2. Secretome Analysis of Umbilical Cord-Derived Mesenchymal Stem Cell Culture Medium

To analyze the secretome of the umbilical cord-derived mesenchymal stem cell culture medium obtained in Preparation Example 1, RayBio Human Cytokine/Growth Factor Antibody (RayBiotech, Noncross, GA, USA) was used to confirm the components of the umbilical cord-derived mesenchymal stem cell culture medium in a serum-free state.

The array membrane was incubated in blocking buffer at room temperature for 30 minutes, followed by treatment with 2 ml of the umbilical cord-derived mesenchymal stem cell culture medium for 1 hour. The membrane was washed five times, followed by treatment with a biotin-conjugated antibody at room temperature for 1 hour to 2 hours, and 2 ml of HRP-conjugated streptavidin as a substrate was added. After 2 hours, treatment with detection buffer was performed for 2 minutes, and the components of the umbilical cord-derived mesenchymal stem cell culture medium were identified by iBright (CL1000 Imaging system, Thermo Scientific), and the signal intensities were measured using iBright Analysis Software and the results thereof are shown in Table 3 below.

TABLE 3

| No. | Scretome | Signal intensity |
|---|---|---|
| 1 | 6Ckine | 174,765 |
| 2 | Adiponectin/Acrp30 | 991,352 |
| 3 | Angiogenin | 286,416 |
| 4 | Angiopoietin-1 | 767,567 |
| 5 | Angiopoietin-2 | 1,177,517 |
| 6 | Angiopoietin-like 1 | 171,227 |
| 7 | Angiopoietin-like 2 | 449,089 |
| 8 | Angiostatin | 1,206,667 |
| 9 | APRIL | 1,265,256 |
| 10 | Artemin | 115,977 |
| 11 | BD-1 | 109,220 |
| 12 | BAX | 1,157,620 |
| 13 | BMP-2 | 322,421 |
| 14 | BMP-3 | 328,360 |
| 15 | BMP-4 | 562,190 |
| 16 | BMPR-IA/ALK-3 | 369,071 |
| 17 | CCR1 | 355,270 |
| 18 | CCR2 | 444,846 |
| 19 | CCR4 | 368,720 |
| 20 | CCR6 | 505,306 |
| 21 | CCR7 | 1,268,737 |
| 22 | CCR8 | 1,006,611 |
| 23 | CCR9 | 1,179,888 |
| 24 | CD30 Ligand/TNFSF8 | 576,651 |
| 25 | CD40/TNFRSF5 | 309,440 |
| 26 | CD40 Ligand/TNFSF5/CD154 | 677,127 |
| 27 | Csk | 58,885 |
| 28 | CLC | 311,049 |
| 29 | CRTH-2 | 811,665 |
| 30 | CTACK/CCL27 | 921,764 |
| 31 | CXCR1/IL-8 RA | 1,059,082 |
| 32 | CXCR2/IL-8 RB | 1,098,793 |
| 33 | CXCR5/BLR-1 | 83,332 |
| 34 | EDA-A2 | 235,812 |
| 35 | EDG-1 | 291,592 |
| 36 | EG-VEGF/PK1 | 260,574 |
| 37 | Endostatin | 118,195 |
| 38 | ErbB4 | 224,633 |
| 39 | Fas Ligand | 107,145 |
| 40 | FGF Basic | 191,618 |
| 41 | FGF R4 | 457,339 |
| 42 | FGF-9 | 1,290,455 |
| 43 | FGF-10/KGF-2 | 135,985 |
| 44 | FGF-11 | 676,128 |
| 45 | IL-13 1B | 503,247 |
| 46 | GDF3 | 168,268 |
| 47 | GDF5 | 142,367 |
| 48 | GDF9 | 194,609 |
| 49 | GDF11 | 124,444 |
| 50 | GDF-15 | 692,563 |
| 51 | GRO-a | 243,738 |
| 52 | HB-EGF | 1,024,445 |
| 53 | HCR (CRAM-A/B) | 510,677 |
| 54 | HRG1-alpha/NRG1-alpha | 292,956 |
| 55 | IGFBP-3 | 168,895 |
| 56 | IGFBP-6 | 42,955 |
| 57 | IGFBP-rp1/IGFBP-7 | 1,180,327 |
| 58 | Lymphotoxin beta/TNFSF3 | 200,799 |
| 59 | M-CSF | 635,274 |
| 60 | MDC | 438,239 |
| 61 | MIP-1a | 1,267,181 |
| 62 | MIP-1b | 395,078 |
| 63 | MIP 2 | 225,617 |
| 64 | NAP-2 | 187,219 |
| 65 | PF4/CXCL4 | 91,533 |
| 66 | PLUNC | 136,297 |
| 67 | Thrombospondin-1 | 353,456 |
| 68 | TIMP-1 | 1,097,222 |
| 69 | TIMP-2 | 240,351 |
| 70 | TMEFF1/Tomoregulin-1 | 1,040,946 |
| 71 | TRADD | 551,168 |

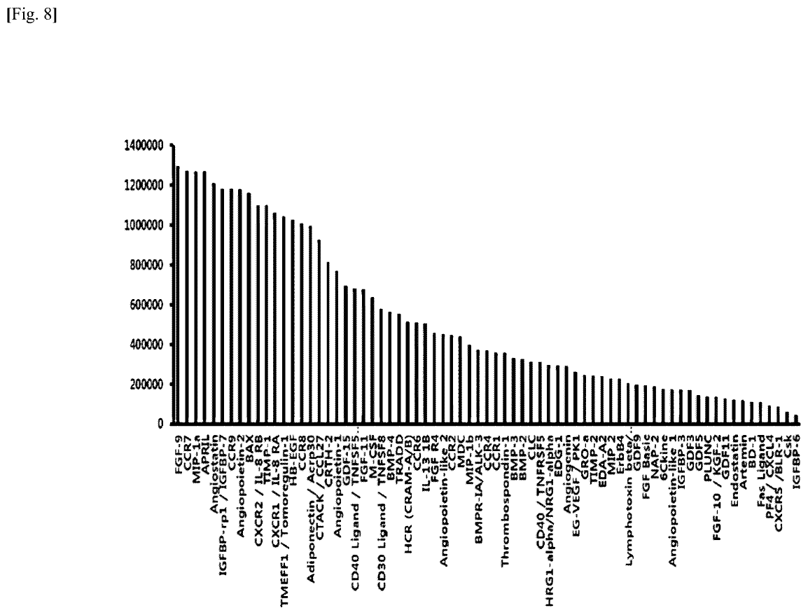

As a result, it was confirmed that the umbilical cord-derived mesenchymal stem cell culture medium contained a large number of various growth factors, cytokines, and the like (FIGS. 7 and 8). Specifically, it was confirmed that the umbilical cord-derived mesenchymal stem cell culture medium contained proteins involved in skin regeneration and skin aging, such as Adiponectin/Acrp30, angiogenin, angiopoietin-1, angiopoietin-2, angiopoietin-like 1, angiopoietin-like 2, angiostatin, bone morphogenetic protein (BMP)-2, BMP-3, BMP-4, bone morphogenetic protein receptor (BMPR)-IA/anaplastic lymphoma kinase (ALK)-3, Csk, CTACK/C-C motif chemokine ligand 27 (CCL27), CXCR2/Interleukin 8 receptor, beta (IL-8 RB), EDA-A2, EDG-1, endocrine-gland-derived vascular endothelial growth factor (EG-VEGF)/PK1, endostatin, ErbB4, basic fibroblast growth factor (FGF Basic), FGF R4, FGF-9, FGF-10/KGF-2, FGF-11, growth differentiation factor (GDF)3, GDF5, GDF9, GDF11, GDF15, GRO-a, heparin-binding EGF-like growth factor (HB-EGF), thrombospondin-1, thioinosine monophosphate (TIMP)-1, TI MP-2, and TMEFF1/Tomoregulin-1.

In addition, it was confirmed that the umbilical cord-derived mesenchymal stem cell culture medium contained proteins necessary for anti-inflammatory effects and the prevention of autoimmune diseases, such as CXCR1/IL-8 RA, C-X-C chemokine receptor type 5 (CXCR5)/BLR-1, endothelial differentiation gene (EDG)-1, Fas Ligand, IL-13 1B, heme-controlled repressor (HCR) (CRAM-NB), macrophage colony stimulating factor (M-CSF), MDC, macrophage inflammatory proteins (MIP)-1a, MIP-1b, MIP-2, neutrophil activating protein (NAP)-2, platelet factor (PF)4/CXCL4, palate, lung, and nasal epithelium clone protein (PLUNC), tumor necrosis factor receptor type 1-associated DEATH domain protein (TRADD), and the like.

### Experimental Example 3. Evaluation of Cytotoxicity and Cell Proliferation Effect of Umbilical Cord-Derived Mesenchymal Stem Cell Culture Medium

To evaluate the cytotoxicity and cell proliferation effect of the umbilical cord-derived mesenchymal stem cell culture medium obtained in Preparation Example 1, the following experiments were carried out using human epidermal cells (HaCaT) and human dermal fibroblasts (HS68).

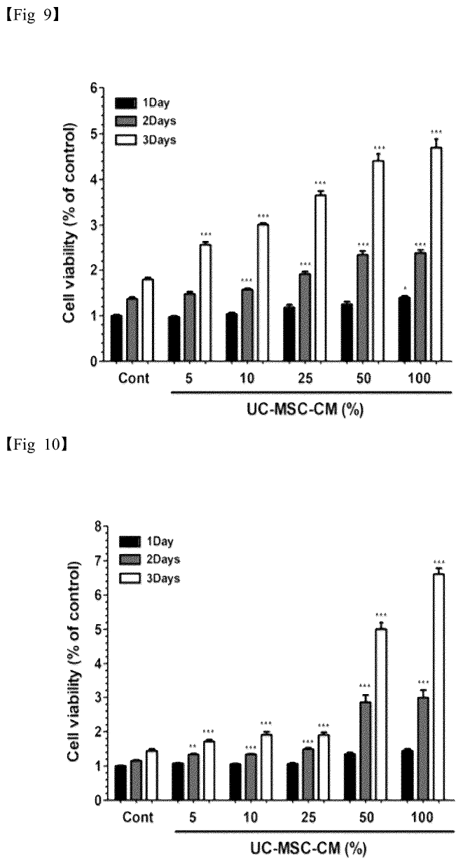

Each of HaCaT and HS68 was seeded into a 96-well plate at a density of 1×10³ cells/10 μl and cultured for 24 hours, and then was not treated as a negative control (N.C; an untreated group) and treated with the umbilical cord-derived mesenchymal stem cell culture medium at concentrations of 5%, 10%, 25%, 50%, and 100% as experimental groups. After treatment with each concentration of the culture medium, changes in cell activity were observed by measuring absorbance at 450 nm using a CCK-8 (Dojindo, CK04-13) reagent at the same time every day for 3 days. As a result, it was confirmed that the viability of HaCaT (FIG. 9) and HS68 (FIG. 10) increased in a manner dependent on the concentration of the treated umbilical cord-derived mesenchymal stem cell culture medium.

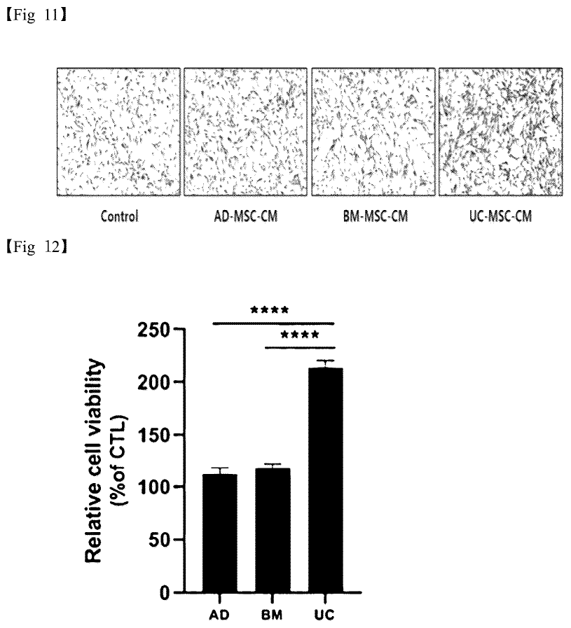

In addition, HaCaT was seeded into a 96-well plate at a density of 1×10³ cells/100 μl per well and cultured for 24 hours, and then was not treated as a negative control (N.C)), and treated with: the adipose-derived mesenchymal stem cell culture medium and bone marrow-derived mesenchymal stem cells obtained in Comparative Example 1 as comparative controls; and the umbilical cord-derived mesenchymal stem cell culture medium at a concentration of 100% as an experimental group, and after 3 days, the cell morphology was observed under a microscope (FIG. 11), and changes in cell activity were observed using a CCK-8 reagent. As a result, it was confirmed that, compared to the cells treated with the adipose-derived mesenchymal stem cell culture medium and the bone marrow-derived mesenchymal stem cell culture medium, the viability significantly increased in the cells treated with the umbilical cord-derived mesenchymal stem cell culture medium (FIG. 12).

Through these results, it can be confirmed that the umbilical cord-derived mesenchymal stem cell culture medium according to an embodiment of the present disclosure has no cytotoxicity and induces cell proliferation.

### Experimental Example 4. Confirmation of Skin Wound Recovery Effect of Umbilical Cord-Derived Mesenchymal Stem Cell Culture Medium

To evaluate the cytotoxicity and cell proliferation effect of the umbilical cord-derived mesenchymal stem cell culture medium obtained in Preparation Example 1, the following experiments were carried out using human epidermal cells (HaCaT) and human dermal fibroblasts (HS68).

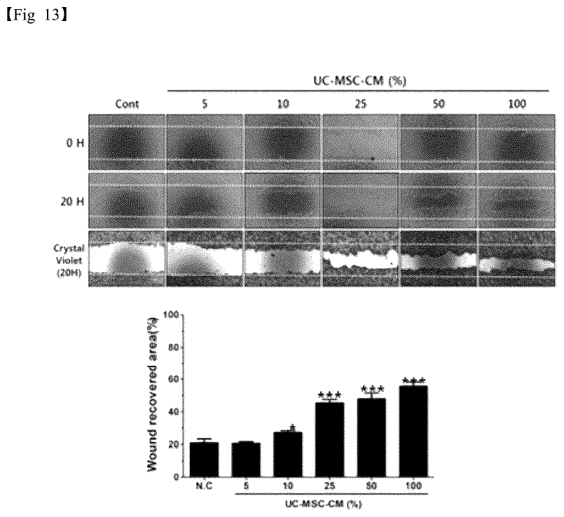

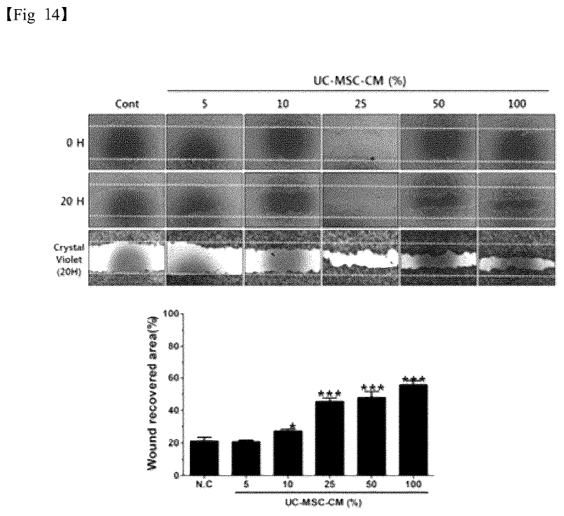

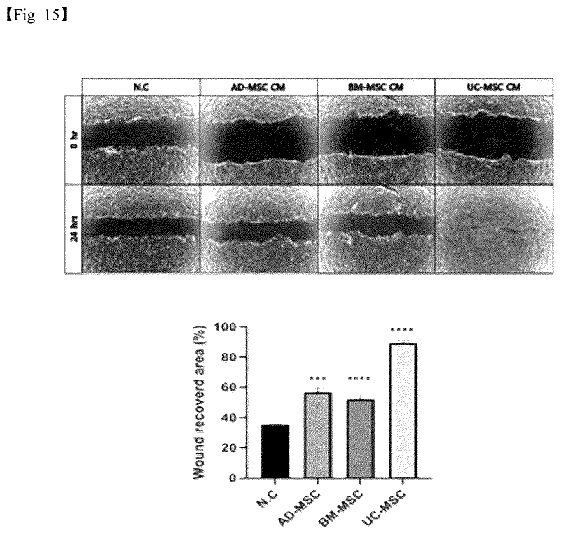

HaCaT was seeded into a 24-well plate at a density of 3×10⁵ cells/well and HS68 was seeded into a 24-well plate at a density of 2×10⁵ cells/well, and then cultured to a confluency of 100%. A wound was made on the cells by scratching the center of each well by using a 1000P white tip, and then each of HaCaT and HS68 was not treated as a negative control (N. C) and treated with the umbilical cord-derived mesenchymal stem cell culture medium at concentrations of 5%, 10%, 25%, 50%, and 100% as experimental groups. Immediately after and 24 hours after each of HaCaT and HS68 was treated with the culture medium, the area of the wound was measured to confirm a recovery rate. At this time, 24 hours after treatment with the culture medium, the cells were stained with a crystal violet reagent and observed under a microscope. As a result, it was confirmed that, when HaCaT (FIG. 13) and HS68 (FIG. 14) were treated with the umbilical cord-derived mesenchymal stem cell culture medium at a concentration of 10% or more, the wound recovery rate was statistically significantly increased.

In addition, HaCaT was seeded into a 24-well plate at a density of 3×10⁵ cells/well and cultured to a confluency of 100%, and a wound was made using the same method as that described above, and then HaCaT was not treated as a negative control (N. C; an untreated group) and treated with: the adipose-derived mesenchymal stem cell culture medium and bone marrow-derived mesenchymal stem cells obtained in Comparative Example 1 as comparative controls; and the umbilical cord-derived mesenchymal stem cell culture medium at a concentration of 100% as an experimental group. Immediately after and 24 hours after culture medium treatment, the area of the wound was measured to determine the recovery rate, and the cells were stained with a crystal violet reagent and observed under a microscope. As a result, it was confirmed that, compared to the cells treated with the adipose-derived mesenchymal stem cell culture medium and the bone marrow-derived mesenchymal stem cell culture medium, the viability was significantly increased in the cells treated with the umbilical cord-derived mesenchymal stem cell culture medium (FIG. 15).

Through these results, it can be confirmed that the umbilical cord-derived mesenchymal stem cell culture medium according to an embodiment of the present disclosure has a cell wound recovery effect.

### Experimental Example 5. Confirmation of Collagen Synthesis Effect of Umbilical Cord-Derived Mesenchymal Stem Cell Culture Medium

To evaluate the collagen synthesis effect of the umbilical cord-derived mesenchymal stem cell culture medium obtained in Preparation Example 1, the following experiments were carried out using human epidermal cells (HaCaT) and human dermal fibroblasts (HS68).

### Experimental Example 5.1. Analysis of Expression Levels of Collagen Genes by RT-PCR

HS68 was seeded into a 6-well plate at a density of 1.0×10⁵ cells/well and cultured for 24 hours. HS68 was not treated as a negative control (N.C)) and treated with the umbilical cord-derived mesenchymal stem cell culture medium at concentrations of 5%, 10%, 25%, 50%, and 100% as experimental groups, and cultured for 24 hours. Then, to analyze the expression levels of collagen synthesis genes, real-time polymerase chain reaction (qPCR) was used as follows.

Specifically, RNA was extracted using phenol/chloroform. The extracted RNA was reverse transcribed to synthesize cDNA. The expression level of cDNA was analyzed on an Applied Biosystems 700 sequence detection system (Foster City, CA, USA) by qPCR. The primers used are the same as shown in Table 4 below.

TABLE 4

| Collagen | | |
|---|---|---|
| gene name | Forward primer | Reverse primer |
| COL1A1 | GGCGGCCAGGGCTCCGAC | GGTGCCCCAGACCAGGAATT |
| | (SEQ ID NO: 1) | (SEQ ID NO: 2) |
| COL3A1 | TGAAAGGACACAGAGGCTTCG | GAGCCTGGTAAGAATGGTGC |
| | (SEQ ID NO: 3) | (SEQ ID NO: 4) |
| β-actin | TCCTCCCTGGAGAAGAGCTA | AGGAGGAGCAATGATCTTGATC |
| | (SEQ ID NO: 5) | (SEQ ID NO: 6) |

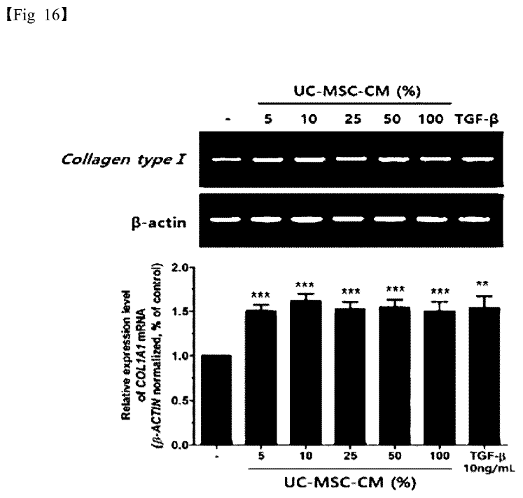

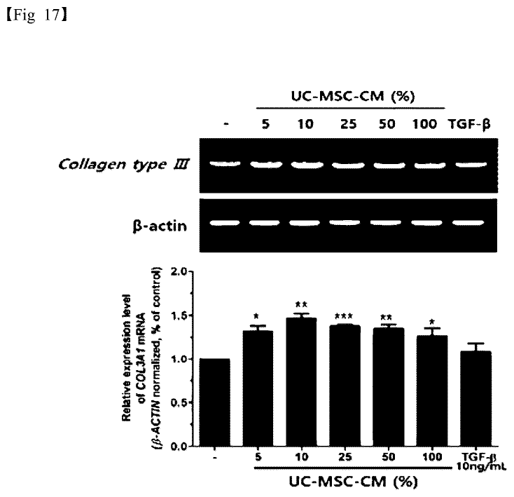

qPCR was repeated 25 times at 95° C. for 10 minutes, 95° C. for 15 seconds and 56° C. for 1 minute. The mRNA levels were normalized to β-actin levels and compared. As a result, it was confirmed that the expression levels of collagen genes COL1A1 (FIG. 16) and COL3A1 (FIG. 17) significantly increased when treated with the umbilical cord-derived mesenchymal stem cell culture medium, compared to the negative control.

### Experimental Example 5.2. Evaluation of Collagen Synthesis Promotion Ability by ELISA

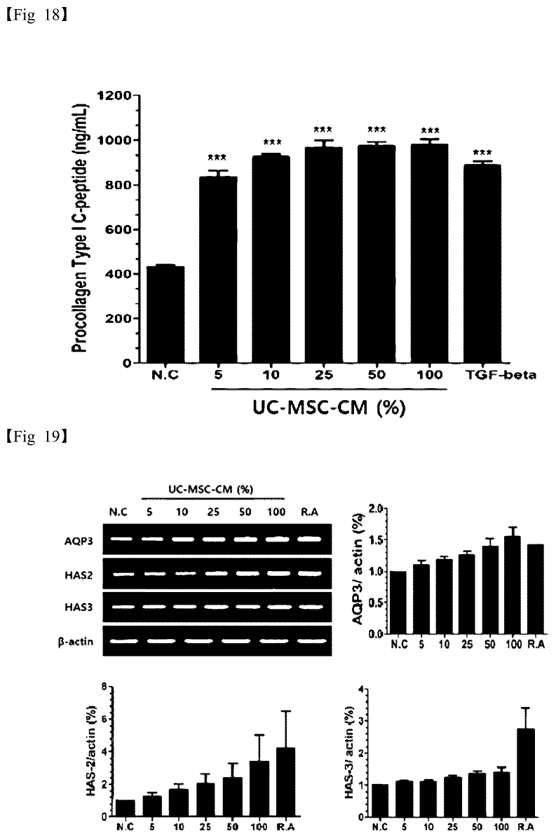

HS68 was seeded into a 6-well plate at a density of 1.0×10⁵ cells/well and cultured for 24 hours. HS68 was not treated as a negative control (N.C) and treated with 10 ng/ml of TGF-β as a positive control, and the umbilical cord-derived mesenchymal stem cell culture medium at concentrations of 5%, 10%, 25%, 50%, and 100% as experimental groups, and cultured for 24 hours. Thereafter, the culture medium was centrifuged to obtain a supernatant. The ability to promote collagen synthesis was confirmed by analyzing the degree of procollagen synthesis by using a Procollagen Type I C-peptide (PICP) ELISA Kit (Takara, Cat. #MK101). As a result, it was confirmed that the expression level of PICP was remarkably increased when treated with the umbilical cord-derived mesenchymal stem cells, and was similar or increased compared to TGF-β, which is previously known to have the ability to promote collagen synthesis (FIG. 18).

Through these results, it can be confirmed that the umbilical cord-derived mesenchymal stem cell culture medium according to an embodiment of the present disclosure has skin wrinkle improvement and skin elasticity increase effects.

### Experimental Example 6. Confirmation of Skin Moisturizing and Barrier Strengthening Effects of Umbilical Cord-Derived Mesenchymal Stem Cell Culture Medium

To evaluate the skin moisturizing and barrier strengthening effects of the umbilical cord-derived mesenchymal stem cell culture medium obtained in Preparation Example 1, the following experiments were carried out using human epidermal cells (HaCaT).

HaCaT was seeded in a 6-well plate at 1.0×10⁶ cells and cultured, followed by replacement with serum-free medium. After 24 hours, HaCaT was not treated as a negative control (N.C), and treated with 1 mM retinoic acid (R.A, Sigma-aldrich, R2625) as a positive control and the umbilical cord-derived mesenchymal stem cell culture medium at concentrations of 5%, 10%, 25%, 50%, and 100% as experimental groups. After 24 hours, RNA was extracted from the cells to synthesize cDNA, and qRT-PCR was performed in the same manner as in Experimental Example 5.1 to analyze the expression levels of aquaporin3 (AQP3), hyaluronic acid synthase (HAS)-2, and HAS-3, which are moisturizing factors. The primers used are the same as shown in Table 5 below.

TABLE 5

| Gene | | |
|---|---|---|
| Name | Forward primer | Reverse primer |
| AQP3 | AGACAGCCCCTTCAGGATTT | TCCCTTGCCCTGAATATCTG |
| | (SEQ ID NO: 7) | (SEQ ID NO: 8) |
| HAS-2 | AGAGCACTGGGACGAAGTGT | ATGCACTGAACACACCCAAA |
| | (SEQ ID NO: 9) | (SEQ ID NO: 10) |
| HAS-3 | CTTAAGGGTTGCTTGCTTGC | GTTCGTGGGAGATGAAGGAA |
| | (SEQ ID NO: 11) | (SEQ ID NO: 12) |

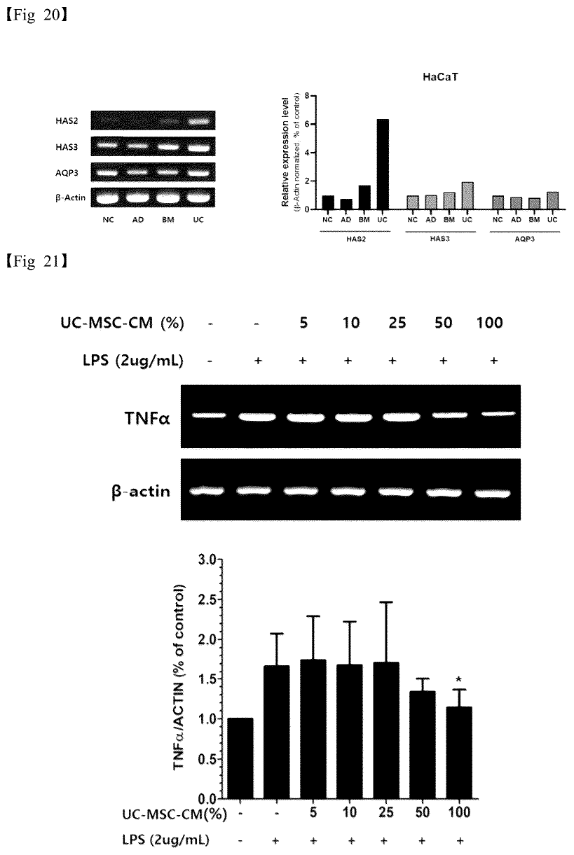

As a result, it was confirmed that the expression levels of AQP3, HAS-2, and HAS-3 were increased when treated with the umbilical cord-derived mesenchymal stem cell culture medium (FIG. 19). In addition, HaCaT was seeded into a 6-well plate at 1.0×10⁶ cells and cultured, followed by replacement with serum-free medium. After 24 hours, HaCaT was not treated as a negative control (N.C), and treated with the adipose-derived mesenchymal stem cell culture medium and bone marrow-derived mesenchymal stem cells obtained in Comparative Example 1 as comparative controls, and the umbilical cord-derived mesenchymal stem cell culture medium at a concentration of 100% as an experimental group. After 24 hours, RNA was extracted from the cells to synthesize cDNA, and qRT-PCR was performed using the same method as the above-described method to analyze the expression levels of AQP3, HAS-2, and HAS-3, which are moisturizing factors. As a result, it was confirmed that, compared to the cells treated with the adipose-derived mesenchymal stem cell culture medium and the bone marrow-derived mesenchymal stem cell culture medium, the expression levels of AQP3, HAS-2, and HAS-3 were increased when treated with the umbilical cord-derived mesenchymal stem cell culture medium (FIG. 20).

The skin performs a barrier function by various moisturizing factors such as hyaluronic acid, and hyaluronic acid is mainly synthesized by HAS of keratinocytes and fibroblasts and accumulated in the extracellular matrix.

Through these results, it can be confirmed that the umbilical cord-derived mesenchymal stem cell culture medium according to an embodiment of the present disclosure has a skin moisturizing effect and a skin barrier strengthening effect therethrough.

### Experimental Example 7. Confirmation of Anti-Inflammatory Effect of Umbilical Cord-Derived Mesenchymal Stem Cell Culture Medium

To confirm the anti-inflammatory effect of the umbilical cord-derived mesenchymal stem cell culture medium obtained in Preparation Example 1, the following experiments were performed using murine macrophages (Raw 264.7; ATCO®, TIB-71™).

Raw 264.7 was seeded into a 6-well plate at a density of 2.5×10⁵ cells/well and cultured to a confluency of 80%, followed by replacement with serum-free medium. After 24 hours, to induce inflammatory responses, Raw 264.7 was treated with 20 μg/mL of lipopolysaccharide (LPS), and was not treated as a negative control (N.C) and treated with the umbilical cord-derived mesenchymal stem cell culture medium at concentrations of 5%, 10%, 25%, 50%, and 100% as experimental groups.

After 24 hours, RNA was extracted from the cells to synthesize cDNA, and qRT-PCR was performed in the same method as in Experimental Example 5.1 to analyze the expression level of TNF-α, which is an inflammatory cytokine. The primers used are the same as shown in Table 6 below.

TABLE 6

| Gene | | |
|---|---|---|
| Name | Forward primer | Reverse primer |
| TNF-α | GCAGGTCTACTTTGGAGTC | CTGGAAAGGTCTGAAGGTAGG |
| | AT (SEQ ID NO: 13) | (SEQ ID NO: 14) |

As a result, it was confirmed that, when inflammatory response-induced cells were treated with the umbilical cord-derived mesenchymal stem cell culture medium, the expression level of TNF-α decreased in a concentration-dependent manner. Through these results, it can be confirmed that the umbilical cord-derived mesenchymal stem cell culture medium according to an embodiment of the present disclosure has the effect of suppressing skin inflammatory responses.

### Experimental Example 8. Confirmation of Antioxidant Effect of Umbilical Cord-Derived Mesenchymal Stem Cell Culture Medium

### Experimental Example 8.1. Measurement of Total Antioxidant Effect

To measure the total antioxidant status of the umbilical cord-derived mesenchymal stem cell culture medium obtained in Preparation Example 1, TAC was measured for a negative control (N.C), the adipose-derived mesenchymal stem cell culture medium at a concentration of 100% and the bone marrow-derived mesenchymal stem cell culture medium at a concentration of 100% obtained in Comparative Example 1 as comparative controls, and the umbilical cord-derived mesenchymal stem cell culture medium at concentrations of 5%, 10%, 25%, 50%, and 100% as experimental groups by the Trolox equivalent antioxidant capacity method as follows.

There are three categories of antioxidants: enzyme systems (GSH reductase, catalase, peroxidase, and the like), small molecules (ascorbate, uric acid, GSH, vitamin E, and the like), and proteins (albumin, transferrin, and the like). Trolox is used to standardize antioxidants and all other antioxidants are measured as Trolox equivalents. Measurement was performed using the Total Antioxidant Capacity Assay Kit, which can measure a combination of small molecule antioxidants and proteins or small molecules alone, and Cu²⁺ ions are converted into Cu⁺ by both small molecules and proteins. Protein Mask prevents Cu²⁺ reduction by proteins, thus enabling the analysis of only small molecules. The reduced Cu⁺ ions are chelated with a colorimetric probe to provide a broad absorbance peak at about 570 nm in proportion to the total antioxidant dose. The colorless reduced form of 2,2′-azinobis(3-ethylbenzothiazo-thiazoline-6-sulfonate (ABTS) is oxidized at acidic pH to blue-green ABTS by hydrogen peroxide. When an antioxidant is present in a sample, ABTS is decolored in proportion to these concentrations, and the result of this color change reaction is measured by irradiation with absorbance at 570 nm. To measure the TAC of a sample material, a standard curve was plotted using Trolox as a standard reagent. Trolox is a typical standard reagent widely used for measuring total antioxidant status, and TAC activity is expressed as Trolox equivalent.

A Cu²⁺ reagent, a sample, and a protein mask were mixed and added into a 96-well plate to make 200 μl, and a reaction was allowed to occur therebetween in an orbital shaker in the dark for 90 minutes, and then measurement was performed by irradiation with absorbance at 570 nm.

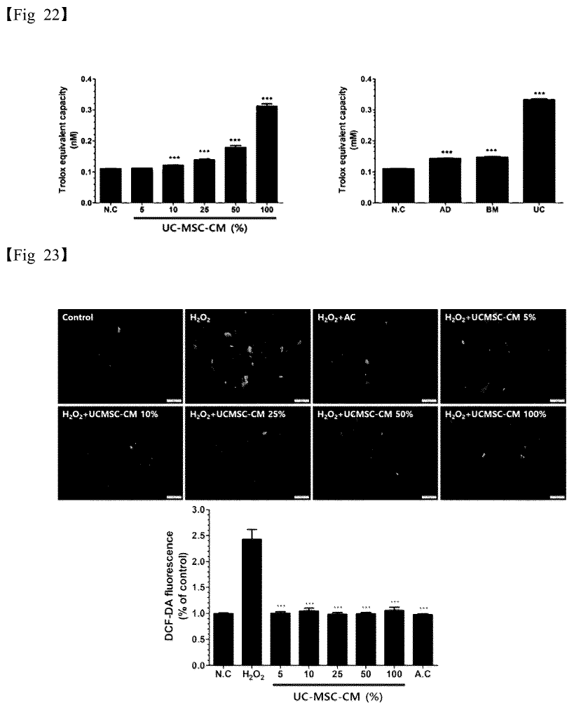

As a result, it was confirmed that, when treated with the umbilical cord-derived mesenchymal stem cell culture medium, the scavenging activity of ABTS radicals was increased in a concentration-dependent manner, and antioxidant substances were significantly increased compared to the cells treated with the adipose-derived mesenchymal stem cell culture medium and the bone marrow-derived mesenchymal stem cell culture medium (FIG. 22).

### Experimental Example 8.2. Scavenging Effect of Intracellular Reactive Oxygen Species (ROS)

To determine the effect of the umbilical cord-derived mesenchymal stem cell culture medium obtained in Preparation Example 1 on the production of intracellular reactive oxygen species (ROS), the following experiments were performed using a carboxy-H2DCFDA-containing ROS detection kit (Abcam).

Dichlorodihydrofluorescin diacetate (DCFH-DA) easily penetrates the cell membrane and diffuses into cells, and is hydrolyzed to DCFH as a result of lost fluorescence by intracellular esterase, and then rapidly oxidized to highly fluorescent DCF in the presence of ROS. Therefore, the fluorescence intensity of DCF is proportional to the amount of ROS in cells.

Human dermal fibroblasts (HS68) were seeded into a 24-well microplate at a density of 2.5×10⁴ cells per well, and cultured in a medium containing 10% FBS and an incubator at 37° C. under 5% CO₂ for 24 hours. Thereafter, each of a negative control (N.C), 250 μM ascorbic acid (Vit. C) as a positive control, hydrogen peroxide as a comparative control, and the umbilical cord-derived mesenchymal stem cell culture medium at concentrations of 5%, 10%, 25%, 50%, and 100% as experimental groups were added, followed by culture for 24 hours.

After 24 hours, 25 μM DCFH-DA was simultaneously added to allow a reaction to occur at 37° C. for 45 minutes, followed by treatment with a 50 μM tert-butyl hydrogen peroxide (TBHP) solution to allow a reaction to occur at 37° C. for 1 minute to 5 minutes. After washing once with 1×PBS, 100 μl of 1×PBS was added to each well, and fluorescence microscopic images were taken, and fluorescence values at an excitation wavelength of 485 nm and an emission wavelength of 528 nm were measured using a fluorescence plate reader.

As a result, it was confirmed that, when the umbilical cord-derived mesenchymal stem cell culture medium was added, the ROS level was significantly lower than that of an oxidative damage-induced group in which the ROS level was increased by hydrogen peroxide (FIG. 23). This means that, by adding the umbilical cord-derived mesenchymal stem cell culture medium in advance, a low level of ROS can be maintained even when exposed to the same concentration of hydrogen peroxide, due to an increase in the activity of an antioxidant system in human dermal fibroblasts.

Through these results, it can be confirmed that the umbilical cord-derived mesenchymal stem cell culture medium according to an embodiment of the present disclosure has a skin antioxidant effect.

1. A method of improving skin condition, comprising administering a composition comprising an umbilical cord-derived mesenchymal stem cell culture medium as an active ingredient to a subject's skin in need thereof.

2. The method of claim 1, wherein the improving skin condition is wound relief, wrinkle improvement, regeneration, elasticity increase, moisturizing, barrier strengthening, anti-inflammation, or antioxidation.

3. The method of claim 1, wherein the umbilical cord-derived mesenchymal stem cell culture medium comprises at least one protein selected from the group consisting of 6Ckine, adiponectin/Acrp30, angiogenin, ANGPT-1, ANGPT-2, ANGPTL-1, ANGPTL-2, angiostatin, APRIL, artemin, BD-1, BAX, BMP-2, BMP-3, BMP-4, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF6/CD154, Csk, CLC, CRTH-2, CTACK/CCL27, CXCR1/IL-8 RA, CXCR2/IL-8 RB, CXCR5/BLR-1, EDA-A2, EDG-1, EG-VEGF/PK1, endostatin, ErbB4, Fas ligand, FGF Basic, FGF R4, FGF-9, FGF-10/KGF-2, FGF-11, IL-13 1B, GDF3, GDF5, GDF9, GDF11, GDF-15, GRO-a, HB-EGF, HCR(CRAM-AB), HRG1-α/NRG1-α, IGFBP-3, IGFBP-6, IGFBP-rp1/IGFBP-7, lymphotoxin-β/TNFSF3, M-CSF, MDC, MIP-1a, MIP-1b, MIP 2, NAP-2, PF4/CXCL4, PLUNC, thrombospondin-1, TIMP-1, TIMP-2, TMEFF1/tomoregulin-1, and TRADD.

4. The method of claim 1, wherein the umbilical cord-derived mesenchymal stem cell culture medium comprises at least one protein selected from the group consisting of adiponectin/Acrp30, ANGPT-1, ANGPT-2, angiostatin, APRIL, CCR7, CCR8, CCR9, CRTH-2, CTACK/CCL27, CXCR1/IL-8 RA, FGF-9, GDF-15, HB-EGF, IGFBP-rp1/IGFBP-7, MIP-1a, and TMEFF1/Tomoregulin-1.

5. The method of claim 1, wherein the umbilical cord-derived mesenchymal stem cell culture medium comprises: at least one protein selected from the group consisting of 6Ckine, ANGPT-2, ANGPTL-1, ANGPTL-2, angiostatin, APRIL, artemin, BD-1, BAX, BMP-3, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF6/CD154, Csk, CLC, CRTH-2, CTACK/CCL27, CXCR1/IL-8 RA, CXCR2/IL-8 RB, CXCR5/BLR-1, EDA-A2, EDG-1, EG-VEGF/PK1, ErbB4, Fas ligand, FGF R4, FGF-9, FGF-10/KGF-2, FGF-11, GDF3, GDF5, GDF9, GRO-a, HCR(CRAM-AB), HRG1-α/NRG1-α, IGFBP-rp1/IGFBP-7, lymphotoxin-3/TNFSF3, M-CSF, MDC, MIP-1a, MIP-1b, MIP 2, NAP-2, PF4/CXCL4, PLUNC, TMEFF1/tomoregulin-1, and TRADD; and
at least one protein selected from the group consisting of 6Ckine, adiponectin/Acrp30, angiogenin, ANGPT-1, ANGPT-2, ANGPTL-1, ANGPTL-2, angiostatin, APRIL, artemin, BD-1, BAX, BMP-2, BMP-3, BMP-4, BMPR-IA/ALK-3, CCR1, CCR2, CCR4, CCR6, CCR7, CCR8, CCR9, CD30 ligand/TNFSF8, CD40/TNFRSF5, CD40 ligand/TNFSF6/CD154, Csk, CLC, CRTH-2, CTACK/CCL27, CXCR1/IL-8 RA, CXCR2/IL-8 RB, CXCR5/BLR-1, EDG-1, EG-VEGF/PK1, ErbB4, Fas ligand, FGF R4, FGF-9, FGF-10/KGF-2, FGF-11, IL-13 1B, GDF11, HCR(CRAM-AB), HRG1-α/NRG1-α, IGFBP-rp1/IGFBP-7, lymphotoxin-3/TNFSF3, M-CSF, MDC, MW-1a, MIP-1b, MIP 2, NAP-2, PF4/CXCL4, PLUNC, TIMP-2, TMEFF1/tomoregulin-1, and TRADD.

6. The method of claim 1, wherein the composition promotes collagen synthesis in skin cells.

7. The method of claim 1, wherein the composition promotes the synthesis of aquaporin or hyaluronic acid.

8. The method of claim 1, wherein the composition inhibits the production of reactive oxygen species (ROS) in skin cells.

9. The method of claim 1, wherein the composition inhibits the production of an inflammatory cytokine in skin cells.

10. The method of claim 9, wherein the inflammatory cytokine is TNF-α.

11. The method of claim 1, wherein an umbilical cord-derived mesenchymal stem cell is:
i) positive for at least one surface antigen selected from the group consisting of CD44, CD73, CD105, and CD90; and
ii) negative for at least one surface antigen selected from the group consisting of CD14, CD19, CD45, and CD34.

12. The method of claim 1, wherein the umbilical cord-derived mesenchymal stem cell culture medium is prepared by a method comprising:
a) isolating mesenchymal stem cells from an umbilical cord from which blood vessels are removed;
b) subculturing the isolated mesenchymal stem cells in serum-free cell culture medium 1 to 10 times; and
c) filtering after a culture medium is obtained in the subculturing.

13. (canceled)


SEQUENCE LISTING (14 sequences)

SEQ ID NO: 1 - DNA, 18 bp, Artificial Sequence
  COL1A1_F
ggcggccagggctccgac

SEQ ID NO: 2 - DNA, 20 bp, Artificial Sequence
  COL1A1_R
ggtgccccagaccaggaatt

SEQ ID NO: 3 - DNA, 21 bp, Artificial Sequence
  COL3A1_F
tgaaaggacacagaggcttcg

SEQ ID NO: 4 - DNA, 20 bp, Artificial Sequence
  COL3A1_R
gagcctggtaagaatggtgc

SEQ ID NO: 5 - DNA, 20 bp, Artificial Sequence
  beta-actin_F
tcctccctggagaagagcta

SEQ ID NO: 6 - DNA, 22 bp, Artificial Sequence
  beta-actin_R
aggaggagcaatgatcttgatc

SEQ ID NO: 7 - DNA, 20 bp, Artificial Sequence
  AQP3_F
agacagccccttcaggattt

SEQ ID NO: 8 - DNA, 20 bp, Artificial Sequence
  AQP3_R
tcccttgccctgaatatctg

SEQ ID NO: 9 - DNA, 20 bp, Artificial Sequence
  HAS-2_F
agagcactgggacgaagtgt

SEQ ID NO: 10 - DNA, 20 bp, Artificial Sequence
  HAS-2_R
atgcactgaacacacccaaa

SEQ ID NO: 11 - DNA, 20 bp, Artificial Sequence
  HAS-3_F
cttaagggttgcttgcttgc

SEQ ID NO: 12 - DNA, 20 bp, Artificial Sequence
  HAS-3_R
gttcgtgggagatgaaggaa

SEQ ID NO: 13 - DNA, 21 bp, Artificial Sequence
  TNF-alpha_F
gcaggtctactttggagtcat

SEQ ID NO: 14 - DNA, 21 bp, Artificial Sequence
  TNF-alpha_R
ctggaaaggtctgaaggtagg